(19) United States
(12) Patent Application Publication
(10) Pub. No.: US 2007/0078404 A1
(43) Pub. Date: Apr. 5, 2007

(54) Safety syringe whose needle can be pulled back

(76) Inventor: Wei-Shui Wu, Taichung Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/186,944

(22) Filed: Jul. 22, 2005

(51) Int. Cl. A61M 5/32; A61M 5/00

(52) U.S. Cl. 604/198; 604/110

(57) ABSTRACT

A safety syringe whose needle can be pulled back, which comprise: an syringe, a rod, a needle seat and a needle sheath, wherein the needle seat is installed inside the container part of the syringe in resist status. After the medicine is injected, the holding part of the push rod will enter the base of the needle seat to make the wall of the base become a conical wall gradually, and then make the holding part and concave-buckle part of the rod connect with the linking part and positioning part of the base. After connection, the outer diameter size of the base is still a little smaller than the inner diameter size of the circular-convex part. Therefore, after using and pulling out the push rod, the needle seat will be departed from the container part and stored inside the syringe to avoid people being stung by the discard syringe.

### BACKGROUND OF THE INVENTION

1. Field of the Invention

The present invention relates to a safety syringe whose needle can be pulled back, and more particularly to a syringe and a needle seat, wherein the needle seat can be stored inside the syringe of the safety syringe after use.

The present invention is the third kind of inventions disclosed patent Registration Ser. No. 10/970,997 which belongs to the same inventor.

2. Description of the Prior Art

A safe medical needle apparatus is helpful for patient, medical personnel, environment and public safety. It is not only involved in dealing with the medical needle apparatus but also can reduce the unsafe injection behavior like: reusing the medical needle apparatus, reusing the needle, or incorrect operation. In order to collect the medical waste, some medical and health facilities discard the medical waste into an anti-seepage, anti-puncture package or an airtight container.

After using a syringe, the needle can be placed into a safe place if a safe medical needle apparatus is used. On the other hand, the safe medical needle apparatus can avoid the needle-stick injuries for medical personnel while they are placing a needle into a needle sheath.

The inventor of the present invention takes the drawbacks of the above-mention safe medical needle apparatus as a lesson and improves their efficient, thereby achieving this safety syringe.

### SUMMARY OF THE INVENTION

The object of this invention is to provide a safety syringe whose needle can be pulled back, which can prevent the pollution of the environment and reduce the volume of medical waste.

Another object of this invention is to provide a safety syringe whose needle can be pulled back, wherein the needle sheath can be stuck in the used syringe.

A yet another object of this invention is to provide a safety syringe whose needle can be pulled back, wherein the needle seat can be placed inside the container part of the used syringe.

A further object of this invention is to provide a safety syringe whose needle can be pulled back, wherein many connection points connect the push rod and the neck, and the push rod and the neck can be broken apart after use.

In order to accomplish the above objects, this invention provides a safety syringe whose needle can be pulled back comprising:

a syringe having a tube room for storing medicine, wherein the top of the tube room is formed a wedge and the bottom of the tube room is set a circular-convex part inside, wherein the circular-convex part and bottom are formed a container part, wherein the inner container part is shrunk into a cone along the bottom from the circular-convex part, and a perforation is set along the outer edge of the syringe in the container part;

a push rod whose top is set a holding part, a concave-buckle part, a blocking part and a neck. Moreover, the push rod is set a collar, and wherein the back end of the blocking part that is below the collar will be wedged in the wedge part and can not be dropped out anymore after the push rod push the tube room of the syringe forward;

a needle seat is set in the container part of the syringe and includes a base with changing wall, a sheath part and a sheath-installation part, wherein the sheath-installation part is set a seal-up component in movable relation. The shape of the lower plane of the base is like an oval shape having a long and short axis, and the total length of the outer surround of the lower plane is smaller than the total length of the inner surround of the circular-convex part, wherein the shape of the upper plane of the base is a circle. The base is a downward concave to set an interlinking part, and the bottom of the interlinking part is set a plurality of positioning parts. There is a hole penetrates through the needle seat, and the hole makes the needle interlink with the hole in the base;

a needle sheath is a cone shape to provide an sheath for the needle seat. The top of the needle sheath is awl-shaped, and the needle sheath can be placed into the tube room through the perforation of the syringe;

said needle seat uses the base to penetrate through the tube room of the syringe, and uses the two ends of the long axis of the base lower plane to prop the lower edge of the circular-convex part, thereby forming a container part inside the syringe in resist status. Moreover, the seal-up component of the needle seat passes through the perforation and props up the bottom of the syringe by the upper plane of the base, and then knit together with the perforation of the syringe, and the collar of the push rod can be knit together with the tube room of the syringe.

### BRIEF DESCRIPTION OF THE DRAWINGS

Other objects and aspects of the present invention will become apparent from the following description of embodiments with reference to the accompanying drawing in which:

FIG. 1 is an exploded view of the first embodiment of the safety syringe whose needle can be pulled back of the present invention;

FIG. 2 is a motion schematic view of the first embodiment of the safety syringe.

FIG. 3 is a motion schematic view of the second embodiment of the present invention.

FIG. 4(A) is an enlarged three-dimensional schematic view of the needle seat of the present invention.

FIG. 4(B) is an enlarged schematic view of the needle seat of the present invention.

FIG. 5(A) is a schematic view showing that after the needle sheath cover the needle seat, the needle sheath cover on the holding part of the push rod; but the positioning part of the needle seat is not positioned completely on the concave-buckle part of the push rod.

FIG. 5(B) is a motion schematic view showing that the needle sheath passes through the perforation of the syringe.

FIG. 5(C) is a schematic view showing that the seal-up component of the needle seat be knit and pushed in the perforation of the syringe, and the seal-up component is set inside the container part of the syringe in resist status at the same time.

FIG. 5(D) is a motion schematic view showing that the push rod can be pulled backward to draw the medicine into the syringe.

FIG. 5(E) is a schematic view showing that the push rod has been pushed forward, and the medicine has been injected, and the holding part of the push rod has entered the needle seat.

FIG. 5(F) is a schematic view showing that after the push rod is pulled backward, the needle seat and the push rod are separated from the container part of the syringe; and

FIG. 6 is a schematic view showing that the push rod props the wedge of the syringe by the blocking part, and make the push rod be broken apart from the neck; then the needle sheath be placed into the syringe through the perforation by the needle sheath's top.

### DESCRIPTION OF THE PREFERRED EMBODIMENT

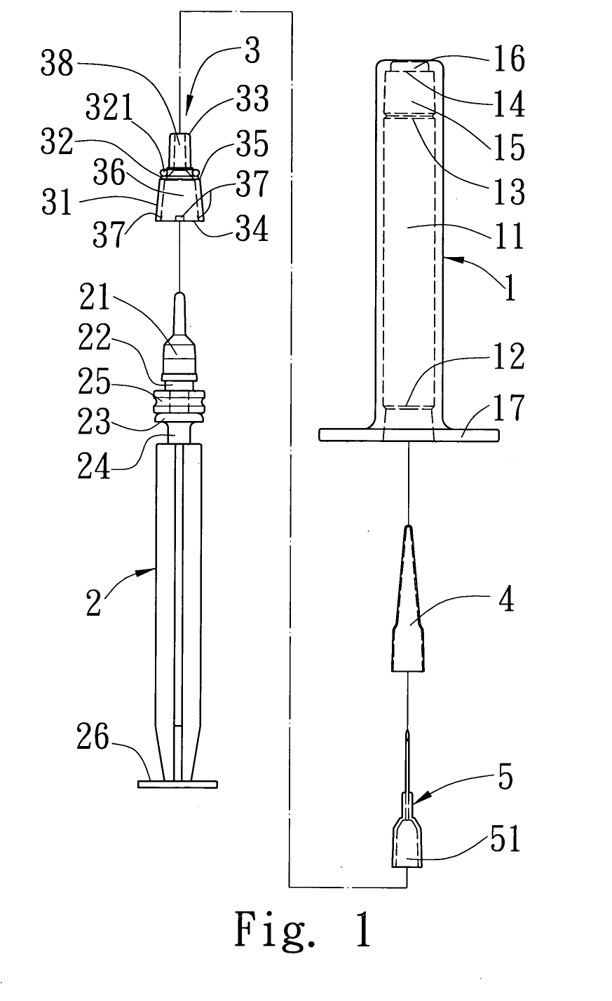

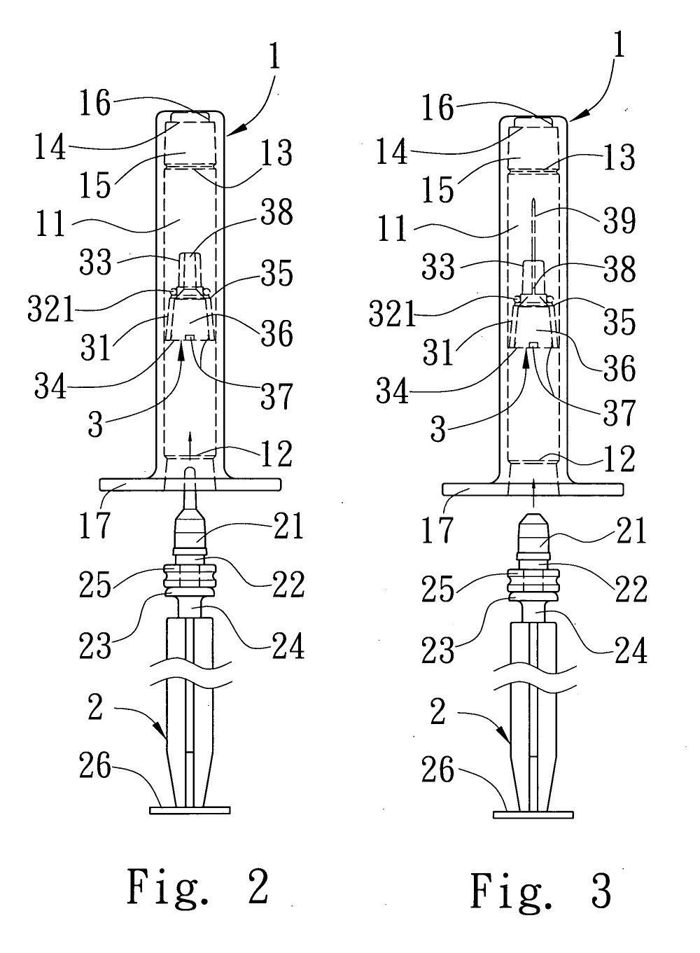

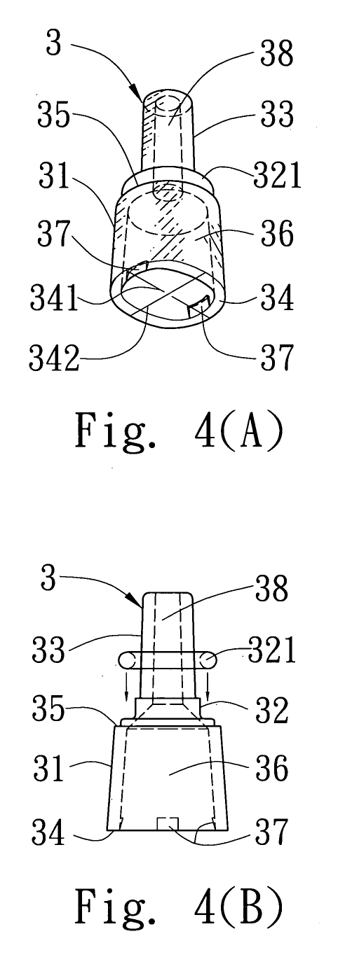

By referencing FIG. 1, FIG. 3, FIG. 4(A) and FIG. 4(B), this invention provide a safety syringe whose needle can be pulled back, which comprise:

A syringe 1 having a tube room 11 for storing medicine, wherein the top of the tube room 11 is formed a wedge 12, and the bottom 14 of the tube room 11 is set a circular-convex part 13 inside, wherein the circular-convex part 13 and the bottom 14 are formed a container part 15, wherein the inner container part 15 is shrunk into a cone along the bottom 14 from the circular-convex part 13, and a perforation 16 is set along the outer edge of the syringe 1 in the container part 15;

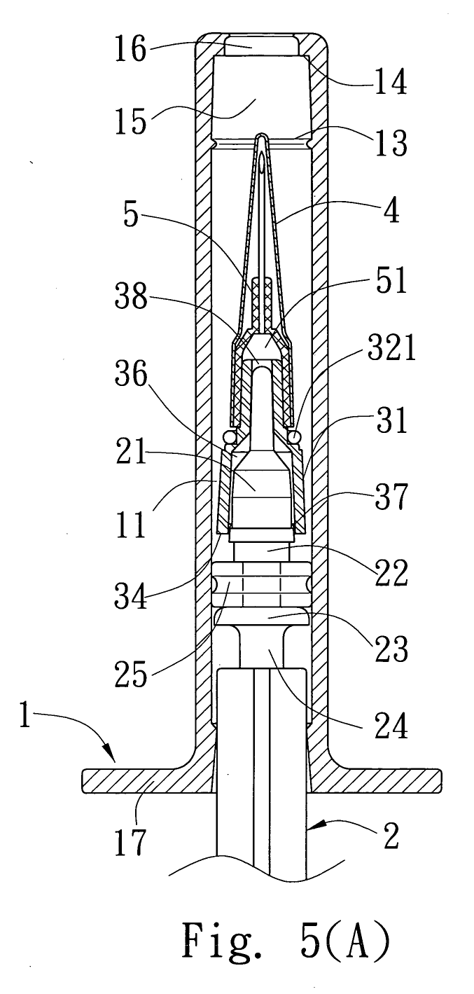

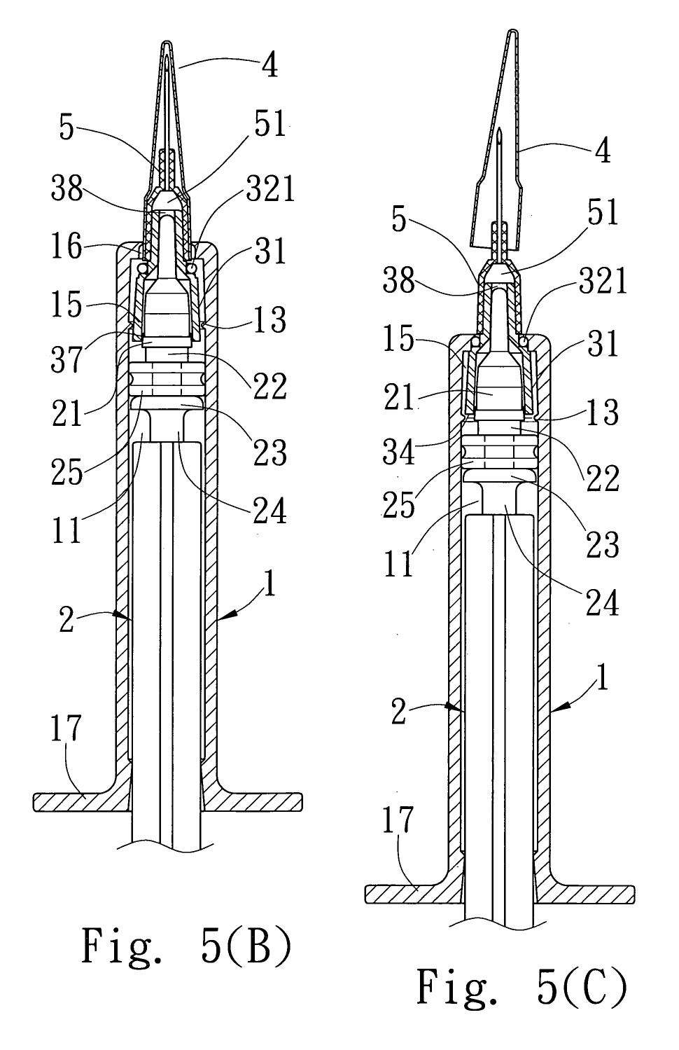

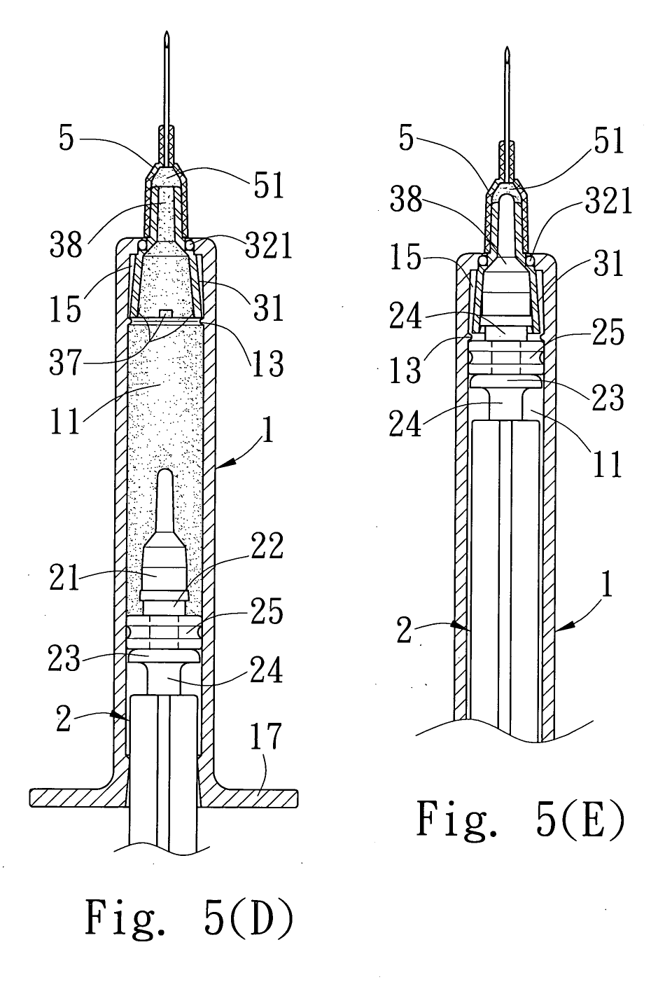

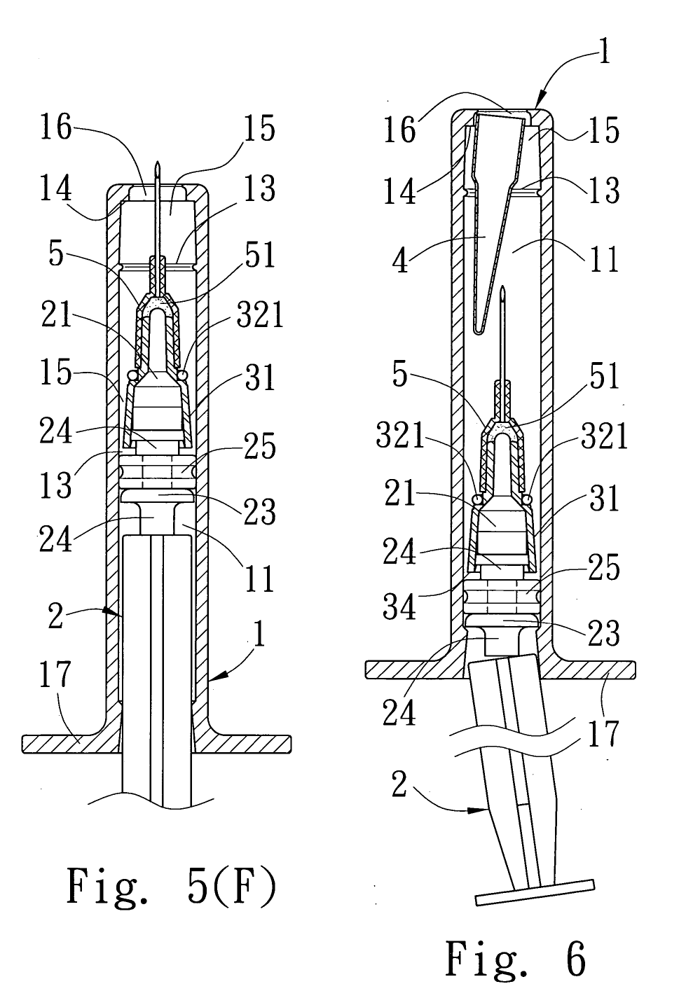

a push rod 2, whose top is set a holding part 21, a concave-buckle part 22, a blocking part 23 and a neck 24, wherein the push rod 2 is set a collar 25, and wherein the back end of the blocking part 23 that is below the collar 25 will be wedged in the wedge 12 and can not be dropped out anymore after the push rod 2 push the tube room 11 of the syringe 1 forward. The FIG. 3 is the second embodiment of the push rod 2 of this invention, and FIG. 5(A) is the third embodiment of the push rod 2 of this invention;

a needle seat 3 set in the container part 15 of the syringe 1, which includes a base 31 with changing wall, an sheath-installation part 32 and a sheath part 33, wherein the shape of the lower plane 34 of the base 31 is like an oval shape having a long axis 341 and short axis 342. The total length of the outer surround of the lower plane 34 is smaller than the total length of the inner surround of the circular-convex 13 part, wherein the shape of the upper plane 35 of the base 31 is a circle. The base 31 is downward concave to set an interlinking part 36, and the top of the interlinking part 36 is set a plurality of positioning parts 37, and the there is a hole 38, which penetrate through the needle seat 3 set on the needle seat 3, wherein the hole 38 can firm up the needle 39 and make the needle 39 to be interlinked with the hole 38 in the base 31 as shown in FIG. 4(A); a needle sheath 4 is a cone shape to provide a sheath for the needle seat 3, wherein the needle sheath 4 is owl-shape and can be placed into the tube room 11 through the perforation 16 of the syringe 1 for reducing the volume of the used syringe, as shown in FIG. 6.

The sheath part 33 of the needle seat 3 is provided for the needle bed 5, and the needle bed 5 is a commonly used standard. However, user can use another standard of needle 39 according to requirement, and then connect the needle bed 5 with the sheath part 33 of the needle seat 3 by the join part 51.

In order to describe this invention further, the schematic views of embodiment of this invention are shown in FIG. 5(A) to FIG. 5(B). While the safety syringe whose needle can be pulled back being used, the collar 25 can be knit with the tube room 11 of the syringe 1 closely because the collar 25 of the push rod 2 is made by rubber or silica gel. Therefore, after the needle sheath 4 covers the needle seat 3, the needle seat 3 covers the holding part 21 of the push rod 2. Moreover, since the positioning part 37 of the needle seat 3 is not positioned completely on the concave-buckle part 22 of the push rod 2, the size of the end of the needle sheath 4 is designed as just can passing through the perforation 16 of the syringe 1. Then user can use first and middle finger to prop the prop part 17 of the syringe 1, and use the thumb to press the press part 26 slightly. At this moment, the needle seat 3 passes through the tube room 11 of the syringe 1 by the base 31, and props the lower edge of the circular-convex part 13 by the two ends of the long axis 341 of the lower plane 34 of the base 31, therefore becomes a container part 15 of the syringe 1 in resist status. Furthermore, the seal-up component 321 of the needle seat 3 passes through the perforation 16 and props up the bottom of the syringe 1 by the upper plane of the base 31, and then knit together with the perforation 16 of the syringe 1, and the seal-up component 321 has the capability to position and guide the needle seat 3 quickly while the seal-up component 321 is entering the syringe 1, as shown in FIG. 5(C).

Subsequently, the needle sheath 4 is separated from the needle seat 3; therefore users can pull the push rod 2 backward for drawing medicine into the syringe 1, as shown in FIG. 5(D). If users press the push rod 2, the push rod 2 will move forward, and the medicine inside the syringe 1 will be injected outside. After the medicine being injected completely, the pressing force make the holding part 21 of the push rod 2 enter the interlinking part 36 of the base 31, and only a few medicine remains inside the join part 51 of the needle bed 5, as shown in FIG. 5(E).

While the holding part 21 of the push rod 2 is entering the base 31, the changing wall of the base 31 become a conical wall gradually to make that the changing wall between the lower plane 34 and the upper plane 35 of the base 31 knit closely with the holding part 21, and thereby the holding part 21 of the push rod 2 is interlinked with the interlinking part 36 of the base 31 by pressing and friction forces. After the holding part 21 of the push rod 2 enters the interlinking part 36 of the base 31, the positioning part 37 of the needle seat 3 is embedded in the concave-buckle part 22 of the push rod 2. At the same time, after the holding part 21 of the push rod 2 enters the base 31, the outer diameter size of the lower plane 34 is a little smaller than the inner diameter size of the circular-convex part 13. Therefore, after user pull out the push rod 2, the needle seat 3 is departed from the container part 15 and stored inside the tube room 11 of the syringe 1 as shown in FIG. 5(F).

In the syringe 1 of this embodiment, the inner diameter of the container 15 is larger than the inner diameter of the perforation 16, thereby forming a step. Therefore, the base 31 can be fit into the container part 15 and the perforation 16 completely when the base 31 is inside the syringe 1. The knit-close design between the container part 15 and the perforation 16 can avoid the medicine being leaking while injecting. Moreover, the wedge 12 located near the prop part 17 of the syringe 1 is formed into a step shape with the top of the syringe 1. Therefore, after the safety syringe whose needle can be pulled back is used, if user pushes the push rod 2 to the wedge 12, the blocking part 23 of the push rod 2 will be locked at the wedge 12 to make the push rod can not be separated from the syringe 1. Furthermore, some interlinking points interlink the push rod 2 and the neck 24, and they can be broken apart after use; moreover, the needle sheath 4 can be placed into the tube room 11 of the syringe 1 through the perforation 16 by the needle sheath's top for reducing the volume of the used syringe, as shown in FIG. 6.

Summing up the above, the present invention not only innovates in space, but also improves much above-mention efficiency to the prior art.

Many changes and modifications in the above-mentioned embodiments of the invention can, of course, be carried out without departing from the scope thereof. Accordingly, to promote the progress in science and the useful arts, the invention is disclosed and is intended to be limited only by the scope of the appended claims.

What is claimed is:

1. A safety syringe whose needle can be pulled back, comprising:
a syringe having a tube room for storing medicine,
wherein the top of the tube room is formed a wedge and the bottom of the tube room is set a circular-convex part inside,
wherein the circular-convex part and the bottom are formed a container part,
wherein the inner container part is shrunk into a cone along the bottom from the circular-convex part, and a perforation is set along the outer edge of the syringe in the container part;
a push rod, whose top set a holding part, a concave-buckle part, a blocking part and a neck,
wherein the push rod is set a collar, and the back end of the blocking part that is below the collar will be wedged in the wedge part and can not be dropped out anymore after the push rod push the tube room of the syringe forward;
a needle seat set in the container part of the syringe, which includes a base with changing wall, an sheath part and a sheath-installation part,
wherein the sheath-installation part is set a seal-up component in movable relation,
wherein the shape of the lower plane of the base is like an oval shape having a long and short axis, and
wherein the total length of the outer surround of the lower plane is smaller than the total length of the inner surround of the circular-convex part,
wherein the shape of the upper plane of the base is a circle. The base is downward concave to set an interlinking part, and the top of the interlinking part is set a plurality of positioning parts, and there is a hole penetrating through the needle seat set on the needle seat,
wherein the hole can firm up the needle and make the needle to be interlinked with the hole in the base;
a needle sheath with a cone shape to provide an sheath for the needle,
wherein the top of the needle sheath is awl-shaped, and the needle sheath can be placed into the tube room through the perforation of the syringe,
wherein the needle seat uses the base to penetrate through the tube room of the syringe, and uses the two ends of the long axis of the lower plane of the base to prop the lower edge of the circular-convex part, thereby forming the container part inside the syringe in resist status, and
wherein the seal-up component of the needle seat passes through the perforation and props up the bottom of the syringe by the upper plane of the base, and then knit together with the perforation of the syringe; moreover, the upper plane of the seal-up component fit the inner perforation tightly, and the seal-up component has the capability to position and guide the needle seat quickly while the seal-up component is entering the syringe, and
wherein the collar of the push rod can be knit together with the tube room of the syringe to provide user can slightly press the press part of the push rod by thumb, and then the medicine inside the syringe will be injected outside from the needle, and after medicine being injected completely, the push force will make the holding part of the push rod enter the interlinking part of the base,
whereby, as the holding part of the push rod is entering the base, the changing wall of the base will become a conical wall gradually to make that the changing wall between the lower plane and the upper plane of the base knit closely with the holding part, and thereby the holding part of the push rod will be interlinked with the interlinking part of the base by pressing and friction forces, and after the holding part of the push rod enter the interlinking part of the base, the positioning part of the needle seat will be embedded in the concave-buckle part of the push rod, and, as the holding part of the push rod enters the base, the outer diameter size of the lower plane 34 is a little smaller than the inner diameter size of the circular-convex part, and then after user pull out the push rod, the needle seat is departed from the container part and stored inside the tube room of the syringe.

2. The safety syringe whose needle can be pulled back according to claim 1,
wherein the needle sheath can covers the needle seat, then the needle seat can covers on the holding part of the push rod, thereby, while the positioning part of the needle seat is not positioned completely, the needle sheath can pass through the perforation of the syringe completely.

3. The safety syringe whose needle can be pulled back according to claim 1,
wherein the needle sheath can be placed into the tube room through the perforation of the syringe by the needle sheath's top.
